(19) United States
(12) Patent Application Publication
(10) Pub. No.: US 2009/0177051 A1
(43) Pub. Date: Jul. 9, 2009

(54) SYSTEMS AND METHODS FOR PROVIDING SUB-DRESSING WOUND ANALYSIS AND THERAPY

(75) Inventors: Edward M. Arons, Passaic, NJ (US); Joel Weiss, Monsey, NY (US)

Correspondence Address: Weiss & Arons, LLP, 1540 Route 202, Suite 8, Pomona, NY, 10970, US

(73) Assignee: Heal-Ex, LLC, Brooklyn, NY (US)

(21) Appl. No.: 12/349,721

(22) Filed: Jan. 7, 2009

Related U.S. Application Data
(60) Provisional application No. 61/019,918, filed on Jan. 9, 2008.
(60) Provisional application No. 61/055,529, filed on May 23, 2008.

(51) Int. Cl. A61F 13/00; A61B 5/00; A61B 19/00

(52) U.S. Cl. 600/306; 602/47; 128/898

(57) ABSTRACT

Thus, systems and methods providing sub-dressing wound analysis and therapy are provided. One embodiment of the invention may include a treatment system including a wound dressing comprising perforations. The system may also include a light receptor and an excitation light source. When the wound dressing is deployed on a wound, the light source may provide light that propagates through the wound and is detectable via the perforations. Furthermore, the light receptor may be configured to receive the transmitted light. The transmitted light may include information relating to the viability of tissue within the wound.

### CROSS-REFERENCES TO RELATED APPLICATIONS

This patent application claims priority from U.S. Provisional Patent Application 61/019,918, entitled “Systems and Methods for Providing Sub-Dressing Wound Analysis and Therapy”, filed Jan. 9, 2008, and from U.S. Provisional Patent Application 61/055,529, entitled “Systems and Methods for Providing Sub-Dressing Wound Analysis and Therapy”, filed May 23, 2008.

### FIELD OF TECHNOLOGY

The present invention relates to apparatus and methods for treating a wound by applying reduced pressure to the wound. Aspects of the disclosure relate more particularly to a wound vacuum. More specifically, aspects of the disclosure relate to systems and methods for promoting advanced wound care using a wound vacuum.

### BACKGROUND OF THE INVENTION

Wounds are often treated by the application of negative pressure using a sealing dressing and a vacuum pump. Negative pressure draws fluid out of a wound, and draws blood into the wound. Both of these effects promote healing.

Wounds at various stages of the healing process may include one or more different types of tissue, such as necrotic tissue, devitalized tissue and healthy tissue. The healing of each type of tissue may be promoted by the application of an appropriate therapeutic agent, such as antibiotic, analgesic and/or tissue-digesting enzyme. Agents that may promote healing of one type of tissue may inhibit healing of another type of tissue in the same wound. Moreover, the spatial distribution of the different types of tissue in the wound may change during the application of negative pressure. Administration of tissue-specific therapeutic agents after the initial placement of the dressing may therefore require removal of the dressing to assess requirements for further administration of therapeutic agents.

It would be desirable, therefore, to provide apparatus and methods to administer a therapeutic agent, without breaching a dressing, to a portion of a wound for which the therapeutic agent is appropriate.

It also would be desirable, therefore, to provide apparatus and methods for identifying, without breaching the dressing, a portion of a wound for which administration of the therapeutic agent is appropriate.

### SUMMARY OF THE INVENTION

It may be desirable to increase the sub-dressing imaging and analysis of wounds in order to more accurately treat a wound patient.

It may be desirable as well to provide an apparatus and method that is adapted to advance the use of a vacuum-assisted wound closure system that simplifies and assures that proper debridement takes place in concert with the use of the vacuum-assisted wound closure system.

Modified vacuum-assisted closure systems and methods according to the invention—which may include a permeable pad placement on or in the wound and a wound drape for sealing enclosure of the pad-may be modified to include systems and methods for introduction to the wound of a tissue-digesting enzyme. The systems and methods according to the invention may further include a light provider, whereby a desired wavelength of light may be directed into and about the wound site. The permeable pad may be placed in fluid communication with a vacuum source for promotion of healing as is known in the art.

Systems and methods involving negative pressure wound treatment are described in U.S. Pat. No. 6,994,702 which is hereby incorporated by reference herein in its entirety.

According to the preferred embodiment of the present invention, the permeable pad preferably comprises a highly reticulated, open-cell polyurethane or polyether foam for good transport of wound fluids while under suction. In some embodiments of the invention, the permeable pad may include gauze, glass or polymer beads, foam or a combination of thereof.

Finally, many other features, objects and advantages of the present invention will be apparent to those of ordinary skill in the relevant arts, especially in light of the foregoing discussions and the following drawings and exemplary detailed description.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows a portion of a conventional negative pressure wound treatment device;

FIG. 2 shows a partial cross-sectional view of a wound treatment device according to the invention;

FIG. 3 shows a schematic cross-sectional view of a valve taken from line A-A of FIG. 2 according to the invention;

FIG. 4 shows a schematic cross-sectional view of the valve taken from line B-B of FIG. 3 according to the invention;

FIG. 5 shows a schematic partial cross-sectional view of an embodiment of an optical device for use in accordance with the principles of the invention;

FIG. 6 shows a schematic top plan view of a wound as divided into regions in accordance with the principles of the invention;

FIG. 7 shows a flow diagram of an exemplary method in accordance with the principles of the invention;

FIG. 8 shows a partial cross-sectional view of a capacitance-based wound analysis device in accordance with the principles of the invention; and

FIG. 9 shows a flow diagram of an exemplary method in accordance with the principles of the invention.

### DETAILED DESCRIPTION OF THE INVENTION

In the following description of the various embodiments, reference is made to the accompanying drawings, which form a part hereof, and in which is shown by way of illustration various embodiments in which the invention may be practiced. It is to be understood that other embodiments may be utilized and structural and functional modifications may be made without departing from the scope and spirit of the present invention.

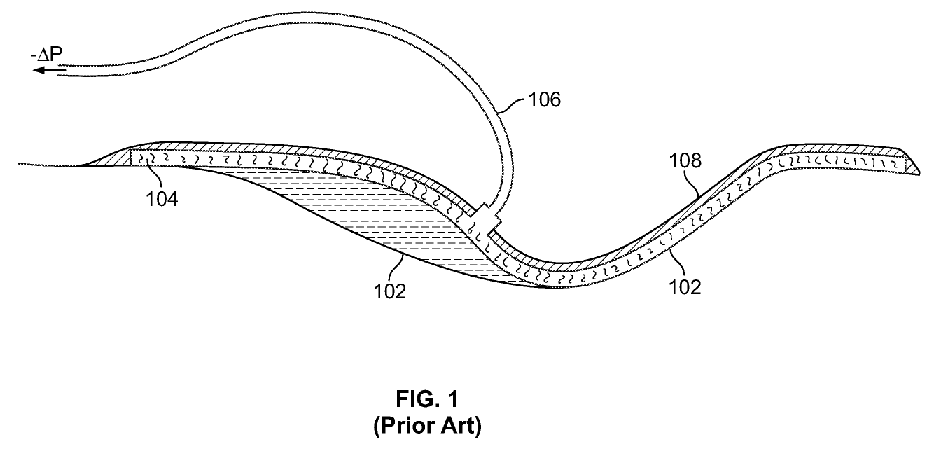

FIG. 1 shows conventional wound treatment device 100 for treating wounds, such as wound 102, using negative pressure—i.e., pressure that is below surrounding (ambient) pressure. A device such as that partially shown in FIG. 1 may include foam sponge 104, which may be formed from a polymer, such as polyester, or any other suitable material. Foam sponge 104 may be in gaseous and/or fluid communication with a vacuum pump (not shown) via a hollow tube such as tube 106. Such a device may be used concurrently with an adhesive sheet 108 which may be used to form a substantially gas-tight and/or fluid tight seal around the sponge such that suction on the sponge via tube 106 may cause negative pressure in the wound. Such a negative pressure device is often used at negative pressures of about 125 millimeters Hg.

Wound tissue may be classified in any suitable manner. Table 1 shows one possible classification.

TABLE 1
An Illustrative Wound Tissue Classification Scheme

| Tissue Type | Characteristics |
|---|---|
| Infected | Associated with increased presence of fluid |
| | Possibly elevated temperature |
| Necrotic | Degenerative cell membranes |
| | No blood flow |
| | Presence of CH, CH2, CH3 and CO bonds with |
| | known optical spectra |
| | Presence of cholesterol |
| Devitalized | Weak profusion |
| | Color more pale than profused tissue-can have |
| | white regions or hues |
| Healthy (granulating) | Perfused |
| | Red |
| Fat | Yellow |
| | Blotchy |
| Blistered | Associated with increased presence of fluid |

The systems and methods shown and described herein may be used to obtain electrical signals based on optical, acoustic, dielectric and/or thermal signals from tissues such as those identified in Table 1. Temporal changes in physical properties in a region of a wound may be tracked. One way to track such a change is to compare a signal at a time t after wound presentation to the signal at presentation (t=0). A photographic image of the wound may be taken at t=0, before application of a dressing. Signals may thus be linked to tissue types that are assigned to the locations based on the image (or based on visual inspection). Changes in tissue classification during a therapeutic regimen may be expressed in measurable changes in physical properties of the tissue.

Table 2 shows illustrative scenarios in which the signals may be used to track changes in wound tissue.

TABLE 2
Illustrative Changes in Wound Tissue Classification

| Tissue Type at | Possible Subsequent Type That Would Lead to a |
|---|---|
| Wound Presentation | Change in Physical Properties |
| Infected | Healthy |
| | Necrotic |
| Necrotic | Healthy (i.e., healthy tissue may |
| | replace enzymatically debrided necrotic tissue) |
| Devitalized | Necrotic |
| | Infected |
| Healthy (granulated) | Devitalized |
| | Infected |
| Fat | Infected |
| Blistered | Healthy |
| | Infected |

Table 3 shows illustrative physical properties that may be used to track changes in wound tissue. When the changes are observed separately in small regions of the wound, temporal changes may be observed in the region and the spatial distribution of the different types of wound tissue may be mapped and tracked during a therapeutic regimen. For example, the distribution of necrotic tissue may be observed over time.

TABLE 3
Illustrative Physical Properties and applications

| Category of probe | Physical characteristic | Tissue Information |
|---|---|---|
| Dielectric | Capacitance | Structure relative to other tissues in wound, structure |
| | Resistivity | Structure relative to other tissues in wound, structure |
| Optical | Reflection | Structure relative to other tissues in wound, structure |
| | Transmission | Structure relative to other tissues in wound, structure |
| | Photoacoustics | Perfusion/oxygenation relative to other tissues in wound, |
| | | perfusion/oxygenation |
| | Microspectroscopy | Necrosis relative to other tissues in wound, necrosis |
| | Laser Doppler Imaging | Perfusion relative to other tissues in wound, perfusion |
| | | Presence of blisters (vessication) |
| | | Structure relative to other tissues in wound, structure |
| Acoustic | Temperature/Heat flux | Perfusion |
| Thermal | | Cellular respiration relative to other tissues in wound, cellular |
| | | respiration |
| | | Metabolism relative to other tissues in wound, metabolism |
| | | Infection relative to other tissues in wound, infection |

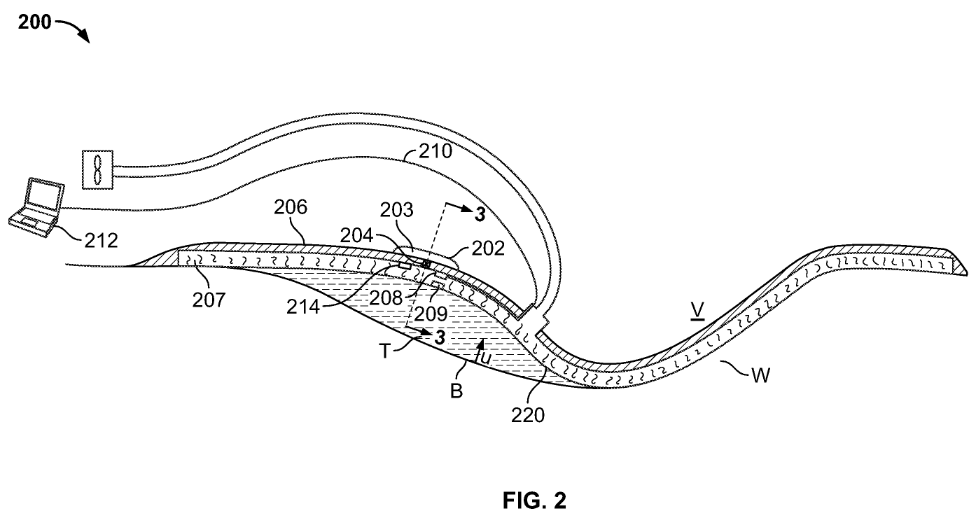

FIG. 2 shows a cross-sectional view of illustrative wound treatment device 200, which is in accordance with the principles of the invention, positioned against wound W. Wound W may include tissue T and void V, from which tissue may have been removed by trauma. Device 200 is preferably a negative pressure wound treatment device and preferably includes bladder 202. Bladder 202 may be adapted to store tissue-digesting enzyme 203, which may be collagenase or another suitable substance. Tissue-digesting enzyme 203 may selectively be released into foam sponge 207 via valve 204, that may be present in sheet 206, which may be configured to adhere to surfaces and may be substantially impermeable to liquids and gases. Enzyme 203 may be in any suitable form, such as a colloid, a gel, an ointment or a liquid. If enzyme 203 is in the form of a gel or an ointment, device 200 may include heating elements to warm the gel or ointment to reduce the viscosity of the gel or ointment and promote absorption by sponge 207. Sheet 206 may be attached to a patient via adhesive.

For the sake of clarity, it will be understood that device 200 “overlies,” or is “on top of” wound W. Arrow U, which points generally away from base B of the wound, shows the “up” direction, to which “down” is opposite.

The device may further include optical excitation head 208. Such an optical device may include a device, such as a light-emitting diode, or a laser diode, that is capable of providing light to a wound area underneath the negative pressure dressing. It should be noted that head 208 may be provided at any suitable position relative to sheet 206 or sponge 207. For example, in embodiments in which sheet 206 is translucent, head 208 may be provided above sheet 206. In some embodiments, head 208 may be located at the bottom of sheet 206. In other embodiments, head 208 may be located within or at the bottom of sponge 207, such as at location 209. Such positioning may depend on the optical properties of the particular sponge and/or the type of light provided by the optical head.

The light provided by optical head 208 may preferably be controlled by a computer 212 via wired connection 210 or a wireless connection.

Device 200 may include one or more sensors 214 positioned within the sponge. In some embodiments, sensors 214 may be optical receivers for receiving light reflected from wound W. The sensors may be wide-spectrum or narrow spectrum sensors. In some embodiments, the sensors may include arrays of sensors, such as charge-coupled devices or digital cameras. In some embodiments, the digital cameras may operate in the visible portion of the electromagnetic spectrum. Any suitable optical elements may be included to precondition, focus or couple light prior to receiving it using sensor 214. The amount of light sensed by sensor 214 may be dependent on the position and/or orientation of head 208 within sponge 207 and on the type of light provided by the optical head.

In embodiments in which sensor 214 is positioned above bottom face 220 of sponge 207, optical fiber channels (not shown) may be provided transversely (generally parallel to direction u) across the thickness of sponge 207 to channel light from wound W directly to sensor 214. In some embodiments, sponge 207 may be replaced in whole or in part by translucent beads. The beads may reduce the frequency and/or size of pockets of therapeutic agents, which may attenuate excitation light exiting the wound. The beads may reduce temporal fluctuations in the optical thickness of the permeable layer and allow for interstitial transport of fluids through static channels under a range of pressure boundary conditions. The transport, which would not be affected by material compression (as it would be in a sponge under vacuum), may be linearly proportional to an applied pressure difference. In some embodiments, the beads may be sintered, which may provide light channeling through the permeable layer. Such embodiments may provide added optical coupling between the permeable layer (such as sponge 207) and the wound tissue.

In some embodiments, sensor 214 may be an acoustic sensor for receiving a photoacoustic signal excited by head 208 in a portion of wound W. In other embodiments, sensor 214 may be a temperature monitor, which may be used to monitor the temperature of wound W. Sensor 214 may be placed on the opposite side of, or around the perimeter of, wound W from excitation head 208 to sense forward-scattered light. Sensor 214 may be configured to sense ultraviolet, visible, near-infrared and/or infrared wavelengths. For measurements that require analysis of specific wavelengths, such as microspectroscopy and Doppler methods, sensor 214 may be configured to sense specific wavelengths or may be coupled with a signal processing module for resolving the presence of those wavelengths in a signal.

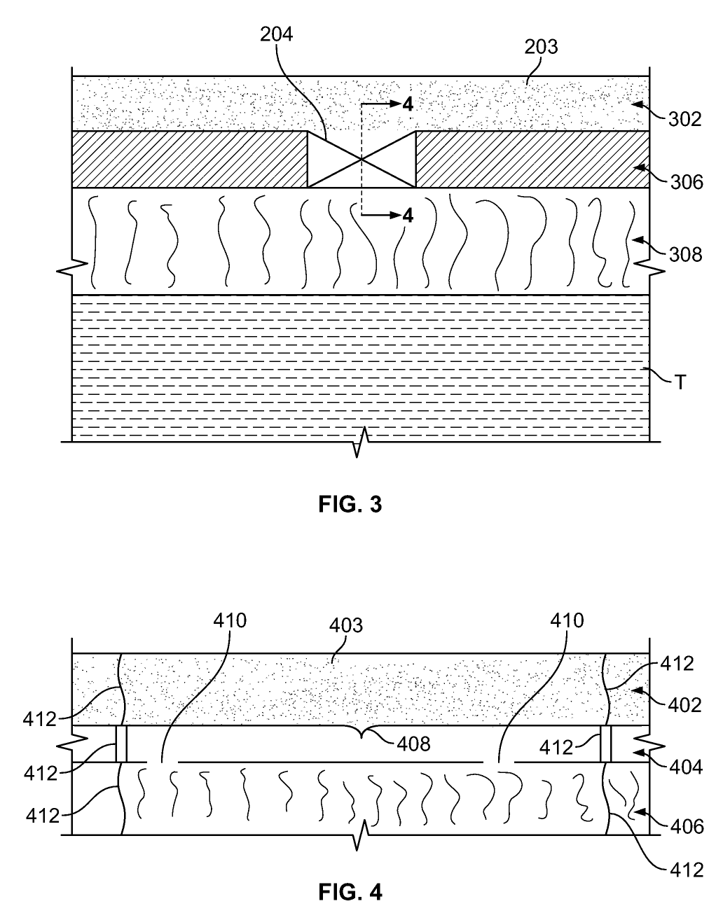

FIG. 3 shows a cross-sectional view of device 200 taken from line A-A of FIG. 2. FIG. 3 shows a schematic diagram of multiple layers, such as layer 302, which is the enclosed area of bladder 202 (shown in FIG. 2). Bladder 202 may be filled with tissue-digesting enzyme 203. FIG. 3 also shows adhesive layer 206, sponge 207 and tissue T of wound W.

Valve 204 is shown schematically within layer 206. It should be noted that valve 304 may also be placed in sponge layer 207 or any other suitable location. Valve 204 may be any suitable valve, including a micro-electric mechanical system that, when opened, allows for the passage of enzyme 203 from bladder layer 302 to sponge layer 207, where the enzyme can be dispersed to an area of tissue T.

FIG. 4 shows a partial cross-sectional view of illustrative device 400, which may have features that correspond to those of device 200 (shown in FIG. 2). The view shown in FIG. 4 corresponds to that taken from line B-B of FIG. 3. FIG. 4 shows bladder layer 402, which may store enzyme 403, adhesive layer 404, and, schematically, valve 408. Valve 408 may allow enzyme 403 to be dispersed from bladder layer 402 to sponge layer 406 through apertures 410.

Dividing walls 412 preferably segregate portions of device 400 such that enzyme can be applied to, and dispersed from, discrete portions of sponge layer 406. While walls 412 are shown as traversing both sheet layer 404 and sponge layer 406, the scope of the invention may also include walls that traverse either one of bladder layer 402, adhesive layer 404, and/or sponge layer 406, or, alternatively, some combination of two of the layers, but not necessarily all three layers.

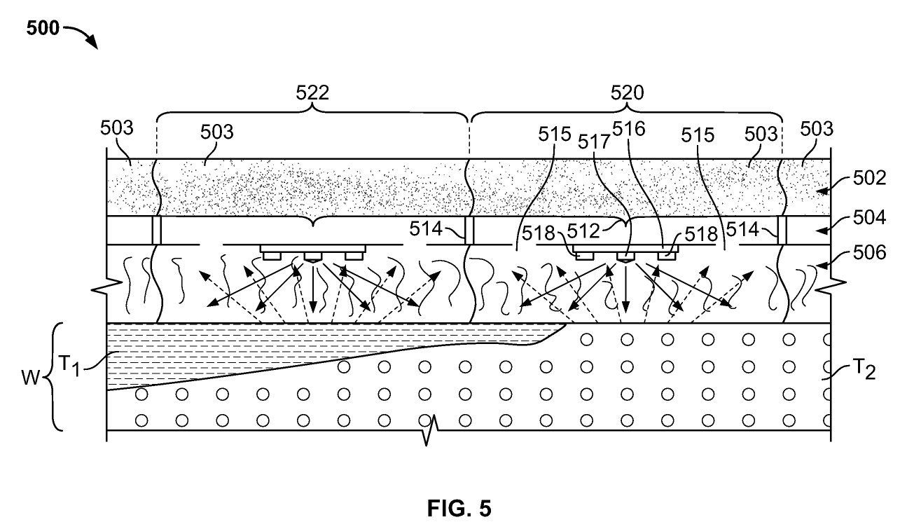

FIG. 5 shows a partial cross sectional view of illustrative device for use according to the principles of the invention. FIG. 5 shows bladder layer 502 that stores tissue-digesting enzyme. Adhesive layer 504 preferably allows (and may regulate) the transmission of enzyme 503 to wound W. Adhesive sheet layer 504 may include valve 512, walls 514 and apertures 515.

Sponge layer 506 (or, alternatively, adhesive sheet layer 504) may incorporate optical head 516. Optical head 516 may include optical device 517. Optical device 517 can preferably produce light at predetermined frequencies, in preferably predetermined spatial and temporal patterns. In one embodiment of the invention, optical device 517 may preferably provide light using a light emitting diode (LED) or other suitable light source.

Optical head 516 may preferably also include optical sensors 518 that sense reflections from the light following transmission of light through sponge layer 506. Such reflections allow sensors to determine the character of wound W in the area underlying sub-dressing wound analysis and treatment cell 520.

Following detection of light information by sensors 518, the information contained in the reflections may preferably be transmitted to a computer at least in order to make some determination regarding the viability of the tissue in the area underlying cell 520. Cell 522 is adjacent cell 520 and can be disposed over a different portion of wound W. It should be noted that, to the extent the cells are disposed over healthy tissue layer T₂, the information in the reflected light should preferably indicate the presence of healthy tissue layer T₂.

In alternative embodiments of the invention, sensors 518 may be disposed in any suitable pattern around, or adjacent to, optical device 517. Furthermore, any suitable number of optical devices 517 and or sensors 518 may preferably be disposed in a single cell.

Other alternative embodiments may include using laser Doppler techniques to determine the viability of the tissue in the area underlying cell 520. Laser Doppler techniques preferably allow for the detection of blood flow through tissue. Because viability of tissue is dependent upon blood flow to an area, such techniques provide a valuable tool for the sub-dressing determination of viability of tissue. In such embodiments, sensors 518 may be tunable to different wavelengths or may include multiple sensors that sense different wavelengths. Sensors 518 may output a signal based on reflection of light from blood flowing through the underlying tissue. The signal may vary with blood flux. The output may be compared, by an analysis module (not shown), to the intensity of reference light reflected from static tissue, such as muscle or fat. The reference light may be obtained below cell 520 or elsewhere.

In yet other embodiments, sensors 518 may be acoustic sensors such as microphones. In such embodiments, the transmission of light and the detection of acoustic waves may be used to form an optoacoustic system that allows for determination of the viability of tissue in the area of the cell. In some optoacoustic embodiments, the acoustic sensors may be placed outside device 500, such as surrounding wound W or on a body surface below (such as on the opposite side of a limb from) wound W.

Yet another technique for determining the viability of tissue may be by determining the temperature of the wound. Because infected tissue typically has a higher temperature than non-infected tissue, the temperature as determined by a temperature sensor within the cell may preferably provide additional information concerning the viability of the tissue.

It should be noted that any of the above-described techniques for determining viability of tissue may be used in conjunction with any of the other techniques in order to obtain a more detailed analysis of the sub-dressing wound condition.

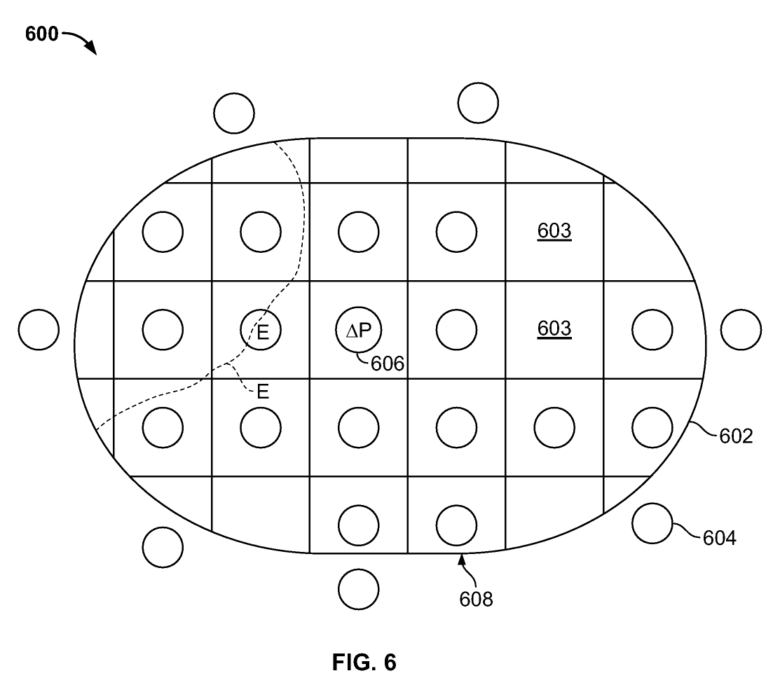

FIG. 6 shows portions of illustrative system 600, which may be used to determine sub-dressing tissue viability using optoacoustic principles. System 600 may include dressing 602, which may overlie wound W (not shown) and be affixed to the body. Dressing 602 may be divided into regions 603 and surrounded by acoustic sensors 604. FIG. 6 also shows negative pressure port 606. The positions of regions 603 and sensors 604 relative to a datum (say, port 606) may be determined by any suitable method, for example, stereotaxis or 3-D mechanical position sensing. Regions 603 may preferably correspond to cells such as 520, 522 as described with respect to FIG. 5.

FIG. 6 shows border D between tissue T₁ (shown in FIG. 5) and tissue T₂. Border D is a surface (not shown) between T₁ and T₂ in wound W. Tissues T₁ and T₂ may have different physical properties that would cause the two tissues to respond differently to similar optical excitation. For example, when T₁ is necrotic tissue, T₁ will be relatively translucent to light in certain wavelengths. For that reason, T₁ will not generate a significant photoacoustic pulse. When T₂ is healthy granulating tissue, T₂ will be is rich in hemoglobin, which will absorb optical excitation close to surface S (not shown) between T₁ and T₂ and convert the excitation into an acoustic pulse. The acoustic pulse may be received by sensors 603.

Known time domain inversion algorithms may be used to determine the location from which the pulses originate based on the time required for the acoustic pulse to propagate through T₂ to the sensors, whose locations are known. The inversion algorithm may be applied individually to each region 603. The height (in direction u, see FIG. 2) of border D under a region 603 may thus be estimated. If T₁ transforms from necrotic to healthy tissue, T₁ will shrink and D will move up. If T₁ becomes infected and causes further necrosis near D, the necrosis can be similarly detected. Although FIG. 6 shows sensors 604 placed peripherally around dressing 602, one or more of the sensors may be placed on the other side of wound W, on the other side of the patients limb or body, opposite dressing 602. Such placement may increase the resolution of estimates of the location of D. In some embodiments, acoustic sensors may be placed within dressing 602 or between dressing 602 and wound W. In such embodiments, necrotic tissue T₁ may attenuate the acoustic signals. The thickness of T₁ may thus be estimated using known inversion algorithms. Inversion algorithms may be used to map wound features based on the electrical signals derived from optical, acoustic, dielectric and/or thermal signals from tissues as shown and described herein.

As shown in FIG. 6, by using detection techniques in a number of cells, a wound area can be defined. In certain embodiments of the invention, prior to treating the wound—i.e., prior to applying a dressing to the wound, or, alternatively, immediately following the application of the dressing in order to obtain a baseline wound reference at T₀ for determining the progress of the wound treatment. Accordingly, the movement of border D may aid the treating physician in determining which regions should be treated with tissue digesting enzyme. In such an embodiment, individual control may be applied to the operation of the valves in each of the cells so that tissue-digesting enzyme may be applied only to the cells that include non-viable tissue.

In some embodiments, an automated control loop based on any known control algorithm, such as a proportional-differential-integral (“PDI”) control algorithm, may be used. In the control algorithm, the thickness of unhealthy tissue, such as T₁, may be treated as a measured variable and it may be controlled by comparison with a desired value such as, in this case: zero thickness.

Using multivariate methods in conjunction with the control algorithm, independent variables such as enzyme delivery rate, delays between enzyme delivery events, saline solution or gas (oxygen or nitrogen, for example) flush rate, delays between flush events, antibiotic delivery rate, delays between antibiotic delivery events, negative pressure magnitude, delays between negative pressure events or any other suitable independent variables may used to optimize the convergence between the measured value and the desired value. The control algorithm may be applied individually to each region 603 or to an index that is based on more than one of regions 603.

Other measured values that may be controlled by the algorithm may include temperature, perfusion indices, oxygenation indices, wound diameter, wound depth, epithelialization indices or any other suitable variables. Scalar indices such as those for perfusion and epithelialization may be established based on measured optical or dielectric signals. For example, where tissue in a region 603 is initially weakly perfused, increasing measured signals from the tissue may be indexed as multiples of the initial signal.

FIG. 6 also shows illustrative perforations 608 that may be resident in dressing 602. Perforations 608 may be distributed throughout the dressing in any suitable distribution—e.g., one perforation per region or many perforations per region—or, in a non-regionalized embodiment of a dressing according to the invention, in some other suitable distribution. One use for perforations 608 may be as follows.

Yet another embodiment of the invention may include placing optical heads on the portion of the body that is opposite the wound—e.g., if the wound is located on the dorsal portion of the arm, the optical heads may be placed on the ventral portion of the arm. In addition, the optical sensors may be provided in or above the area of the wound—i.e., on the dorsal portion of the arm. Such an embodiment may preferably include providing optical heads including an infra-red (“IR”) light source. As such, the optical sensors may be used to determine the presence and/or extent of blood flow through or near the wound, and, consequently the viability of the wound. Thereafter, the condition of the wound can be analyzed based on the transmitted, reflected, or scattered IR light. In fact, the optical heads can be placed at 360 degrees about the wound area because light scatters in all directions.

Such analysis may be performed by known techniques and systems. Such systems may include the light or illumination source, the sensor, and the display. The illumination source may be a matrix of light emitting diodes emitting infrared light. In certain embodiments of the invention, the light source may be directed to the surface of the limb from a distance or applied directly. If applied directly, the light may pass through the limb. Such analysis may be combined with the optoacoustic implementation described above or independently therefrom.

Certain embodiments of the invention may use a dressing, such as a sponge as described herein, as part of a negative pressure wound dressing or other material as described herein. Certain types of dressing material may not transmissive to IR light. Although the dressing, or portions thereof, may block the transmission of the IR light, this may be overcome by either of the following two embodiments of the invention.

First, the dressing may be perforated by perforations 608 whereby certain portions of the dressing are punched through such that the IR light can pass through the thickness of the dressing. As such, the IR light that is transmitted through the sponge can be used to determine the viability of the wound without having to remove the dressing. Further, in certain embodiments of the invention, the portion of the dressing that touches the wound may include a preferably non-adhesive transmissive facing over perforations 608 in order to prevent the wound tissue from migrating up into the dressing while still allowing light to pass through dressing.

Once the IR light has made its way through the tissue, it typically travels in a straight line to the detector. Inside the detector, the IR light is converted into an electrical signal. The electrical signal is then converted to a visible image. Such an embodiment can preferably allow health care professional-supervised debridement to occur while the dressing or other suitable material is maintained in place, and at negative pressure, on the wound.

Second, the optical sensor may be mounted on the side of the dressing that touches the wound. As such, the optical sensor can detect the IR light transmitted through the sponge and provide information regarding the viability of the wound, again, without removing the dressing.

In certain embodiments of the invention, the operator can see a real time image of the subcutaneous vasculature, the distribution of necrotic and devitalized tissue and, consequently, determine the status of the wound in real time.

Because such a system allows blood to be seen in subcutaneous spaces, this can aid in the detection and monitoring of vein trauma. Additionally, vessels in extremities with reduced blood flow can be detected and monitored.

It should be noted that direct illumination is not required to achieve a useful image according to the invention. Rather, the operator may choose to illuminate at nearly any angle from where an image is desired.

A computer such as 212 (shown in FIG. 2) may instruct the valves resident in the cells covering the non-viable tissue to release the tissue-digesting enzyme into the area covered by the cell in order to dispose of the non-viable tissue. In some of those embodiments, preferably no user interaction may be required to administer the sub-dressing treatment by the device. Rather, the control loop may serve to treat the wound until the presence of non-viable tissue is no longer detected.

In some embodiments of the invention, the bladders, such as the bladders shown in FIGS. 2-6, that store collagenase or other appropriate therapeutic agents, including tissue-digesting enzyme, may be replaced by a centralized receptacle of therapeutic agent. Such receptacle may be located remotely from the dressing. Such receptacle may be formed from a flexible polymeric material, rigid polymeric material, or other suitable material.

One embodiment of the centralized receptacle may preferably include placing the receptacle in fluid communication with different regions of the dressing using a number of tubes. The tubes preferably allow for the transport of the therapeutic agent from the receptacle to a selected one, or many, regions of the dressing. In certain embodiments of the invention, the tubes may be bundled into a single tube wherein each of the tubes provides a unique, preferably independently regulated, path from the receptacle to the wound, but the tubes are contained for at least the majority of the length of the tubes within a containing tube.

Such a transport of the tissue-digesting enzyme through tubes from the receptacle to a selected one, or many, regions of the dressing can be regulated by manually-controlled valves, computer-controlled valves, MEMS (micro-electric mechanical systems) or other suitable device. The controlling valves may be located either proximal to the centralized receptacle or proximal (or, alternatively, within) the dressing itself.

In certain embodiments of the invention, the tissue-digesting enzyme may be stored and/or released under pressure, or at an elevated temperature, in order to allow the ointment to flow easily through the tubes to the appropriate regions of the dressing. Alternatively, or in concert with, the embodiment of the invention including storing the tissue-digesting enzyme under pressure, an apparatus according to the invention may preferably include a heating apparatus adapted to heat the tissue-digesting enzyme ointment and, thereby, enhance the flow characteristics of the ointment.

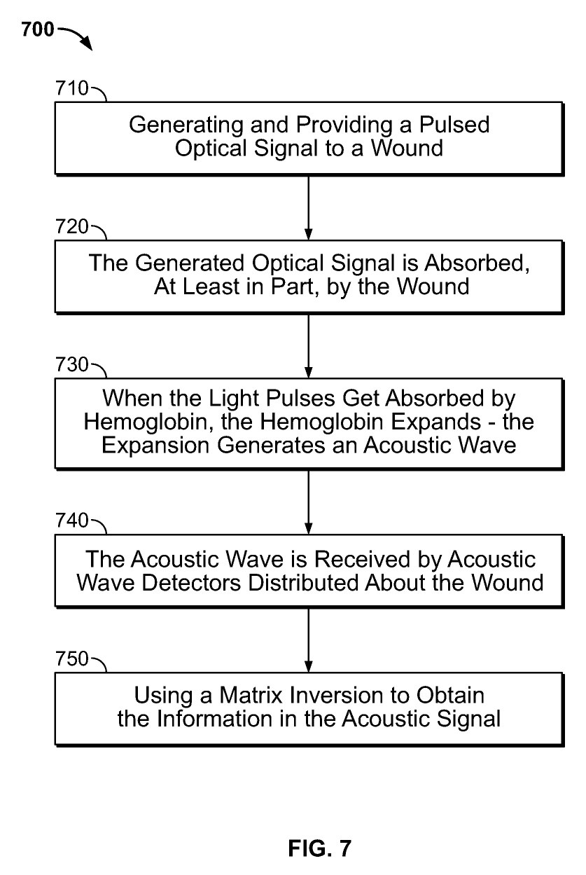

FIG. 7 shows illustrative method 700, for performing wound analysis based on photoacoustic principles in accordance with the principles of the invention. At step 710 shows generating and providing a pulsed optical signal to a wound. Step 720 shows that the generated signal is absorbed, at least in part, by blood in the wound. Thereafter, step 730 shows that when the light pulses get absorbed by hemoglobin, the hemoglobin and surrounding fluid expands. The expansion preferably initiates an acoustic wave. The acoustic wave contains information relating to the viability of the tissue in the wound.

The acoustic wave can be received by acoustic wave detectors—e.g., microphones—that are distributed about the wound, as shown in step 740. Step 750 shows using a matrix inversion to obtain the information in the acoustic signal. The matrix inversion is a known mathematical model that may be used to account for the distance of each receiver from the optical head.

In another embodiment of the invention, the foam or sponge pad may also provided with an optical pigtail, as described in the above-incorporated '702 patent. This optical pigtail may include an optical fiber that has been formed to fan into a plurality of sections of the sponge. The fibers of the most distal fanned sections, which are implanted in the foam pad at its base, are provided with tiny optical slots. The optical slots may preferably be oriented toward the wound site.

Each optical slot can be made by stripping the cladding from the optical fiber in the certain areas of the fanned sections. Such slots form slot radiators. Each of the slot radiators may be adapted to illuminate a portion of the wound site.

The illumination obtained from the slots may be used in place of optical heads. The sensors as described above may preferably use the reflections of the light from the optical slots, or the acoustic waves generated by the light from the optical slots, to determine the viability of the wound tissue.

In another embodiment, glass or polymer beads, which may be loose or sintered, may be used in place of, or in conjunction with, the foam or sponge pad. The beads may be contained in containers that correspond to a region such as 603 (shown in FIG. 6). Each container may have mesh top and bottom walls and impermeable reflective or opaque side walls. The mesh top and bottom walls allow fluid to flow in and out of the containers. The side walls may prevent the flow of fluid from one cell to another and may prevent light from one cell from propagating into another. Beads maintain constant thickness and fluid flow properties under vacuum. The constant thickness may be useful for inversion computations that require known distances between an optical head on top of the beads and the bead-wound interface. The constant fluid flow properties may be useful for controlling administration of therapeutic agents through the beads.

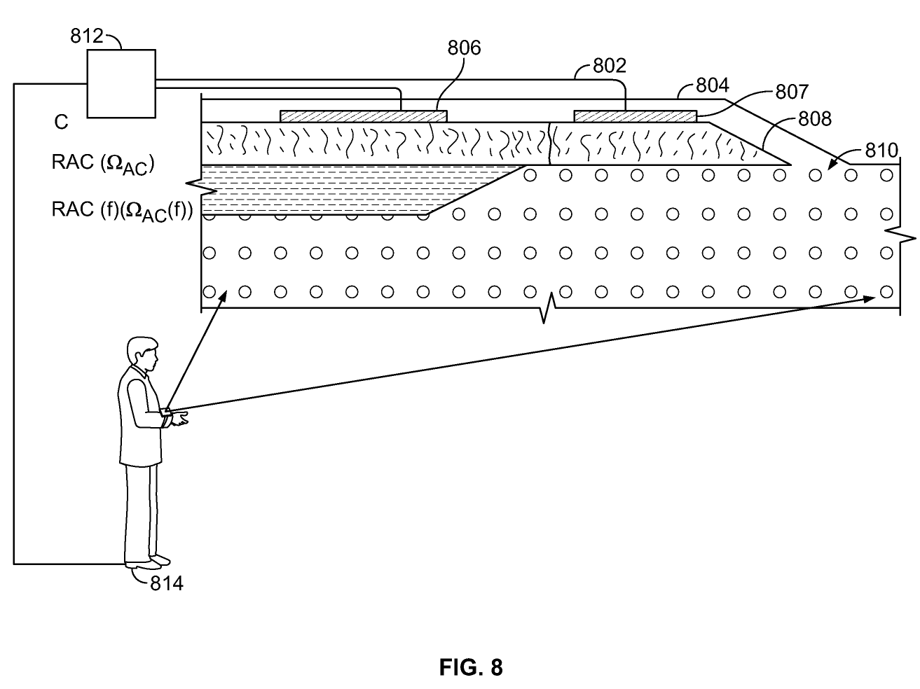

FIG. 8 shows yet another embodiment of a sub-dressing wound detection device. The device preferably includes adhesive sheet 804, electrode 806, electrode 807, sponge 808, computer 812, and electrode 814. The device is preferably adapted to apply a relatively high-frequency signal across electrode pairs (a) 806 and 814; and (b) 807 and 814. Any suitable physical relationship, such as Ohm's law or the relationship between capacitance, charge and voltage, may be employed to measure the resistance or capacitance of the paths between (a) 806 and 814; and (b) 807 and 814. Electrode 814 may be placed at a distance from electrodes 806 and 807 to reduce apparent differences in electrical properties due to differences in the paths' lengths and anisotropy of dielectric properties of different tissues.

In a dielectric-based embodiment that is not shown, closely spaced electrodes may be placed together within a zone such as 603. The electrodes may be used to measure wound tissue resistivity or capacitance at high frequency. Because high frequency signals attenuate rapidly in the wound tissue, the frequency of the excitation signal and the geometry and location of the electrodes may be chosen to target, say, necrotic tissue near the surface of the wound. One possible example is to use concentric electrodes to sense a substantially annular region near the surface of the wound. Parallel elongated electrodes may be used to sense a half-cylinder-shaped region near the surface. Any of the well-known relationships between electrode geometry, electric field shape and resistivity or capacitance may be used to compute resistivity, conductivity, capacitance, or indices thereof for monitoring the healing of a wound below a dressing.

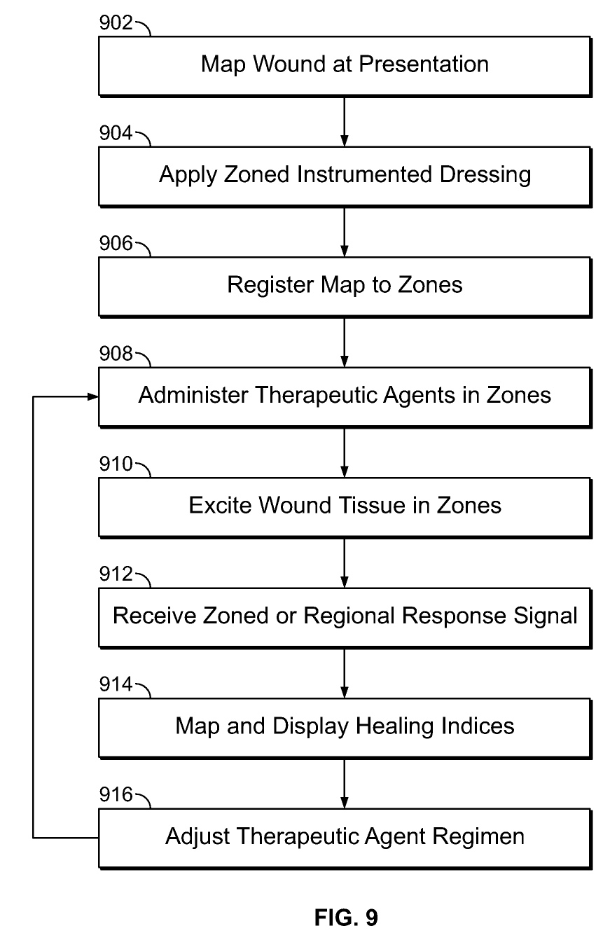

FIG. 9 shows illustrated control process 900, which may involve the use of devices shown and described herein. Process 900, which will be described in relation to wound W, may include step 902, during which a computer-readable initial image may be made of wound W (at t=0). A zoned instrumented dressing such as any of those shown and described herein may be applied to the wound at step 904. At step 906, dressing zones, such as regions 603 (shown in FIG. 6) may be co-registered with identified locations in the wound or skin surrounding the wound. A digital map showing the zones and the initial image may be created. Therapeutic agents may be administered to each of the zones in amounts corresponding to the type of tissue in the zone at step 908. Optical, acoustic, or dielectric excitation may be initiated in each of one or more of the zones at step 910 and a corresponding response received using an appropriate one of the receivers shown and described herein at step 912. The response, after appropriate filtering and signal processing, may be mapped as a layer (corresponding to t=t₁, e.g.) onto the digital map at step 914. At step 916, the regimen of therapeutic agent administration may be altered to achieve desired therapeutic results. The regimen may be altered based on a control algorithm or health care provider judgment. Process 900 may continue at step 908, and further iterations of steps 908-916 may be performed, as appropriate.

Yet another aspect of the invention relates to materials used to form the sponge. One issue that has arisen with respect to negative pressure dressings is that, as the dressing remains on the wound in a negative pressure environment, new tissue may grow into the pores of the sponge. During removal of the dressing, the portion of the issue that has migrated into the sponge must be cut from the wound in order to allow the dressing to be removed from the wound. This cutting away of tissue may have adverse effects on the healing of the wound.

In one embodiment of the invention, the sponge, which, as described above, may be formed with a polymer, such as polyester, or any other suitable material, may further include a layer of photosoluble material. Alternatively, the sponge may be formed completely from a material that is photosoluble.

In such an embodiment of the invention, the partially or wholly photosoluble sponge may be used as part of a negative pressure dressing. When the dressing is removed, the surgeon can cut away the portion of the sponge into which the tissue has not migrated, and then irradiate the portion of the sponge that is bonded to the wound with light from an appropriate portion of the electromagnetic spectrum (which may include light from the Ultra Violet “UV” portion of the electromagnetic spectrum). The applied light may dissolve the portion of the sponge into which the tissue has migrated without any adverse effects on the wound healing process.

Aspects of the invention have been described in terms of illustrative embodiments thereof. A person having ordinary skill in the art will appreciate that numerous additional embodiments, modifications, and variations may exist that remain within the scope and spirit of the appended claims. For example, one of ordinary skill in the art will appreciate that the steps illustrated in the figures may be performed in other than the recited order and that one or more steps illustrated may be optional.

Thus, systems and methods for providing a debriding wound vacuum have been described. Persons skilled in the art will appreciate that the present invention can be practiced by other than the described embodiments, which are presented for purposes of illustration rather than of limitation, and the present invention is limited only by the claims which follow.

What is claimed is:

1. A treatment system comprising:
a wound dressing comprising perforations;
a light receptor; and
an excitation light source;
wherein, when the wound dressing is deployed on a wound:
the light source provides light that propagates through the wound and the light is detectable via the perforations; and
the light receptor for receiving the transmitted light, the transmitted light including information relating to the viability of tissue within the wound.

2. A wound vacuum comprising:
a perforated sponge for application to a wound;
a flexible sheet for covering and sealing the wound;
a plurality of bladders, each of said bladders in fluid communication with a portion of the sponge, each of said bladders containing a debridement substance;
a negative pressure device that applies negative pressure via a tube to the sponge, said negative pressure device being adapted to apply negative pressure via suction through the tube in order form a seal between the flexible sheet and the wound;
a light source for propagating light through the wound; and
an optical receptor for receiving light exiting the wound, the light exiting the wound including information relating to the viability of tissue within the wound, the optical receptor that receives the light exiting the wound via perforations in the sponge.

3. The wound vacuum of claim 2 wherein the perforations in the sponge pass from the face of the sponge that is proximal to the wound to the face of the sponge that is distal from the wound, the apertures of the perforations in the side of the sponge being covered with a transparent, non-adhesive material

4. A method comprising:
deploying a perforated wound dressing on a wound, the wound dressing comprising a light receptor;
propagating light through the wound; and
using the optical receptor to receive light exiting the wound via the perforations, the light exiting the wound including information relating to the viability of tissue within the wound.

5. A wound dressing comprising:
an excitation light source; and
a reflected light receptor;
wherein, when the wound dressing is deployed on a wound:
the light source provides excitation light to the wound;
the light receptor receives reflected light from the wound; and
the reflected light includes information relating to the viability of tissue within the wound.

6. A wound vacuum comprising:
a sponge for application to a wound;
a flexible sheet for covering and sealing the wound;
a plurality of bladders, each of said bladders in fluid communication with a portion of the sponge, each of said bladders containing a debridement substance;
a negative pressure device that applies negative pressure via a tube to the sponge such that gas pressure in the wound is less than gas pressure outside a seal formed by the flexible sheet around the wound;
a light source for providing excitation light to the wound; and
an optical receptor for receiving reflected light from the wound, the reflected light including information relating to the viability of tissue within the wound.

7. A method comprising:
deploying a wound dressing on a wound, the wound dressing comprising a light source and a light receptor;
using the light source to provide excitation light to the wound; and
using the optical receptor to receive reflected light from the wound, the reflected light including information relating to the viability of tissue within the wound.

8. A method for treating a wound comprising:
applying a sponge for application to a wound;
sealing the wound with a flexible sheet;
applying suction to the wound via the tube, the flexible sheet forming a seal around a perimeter of the wound;
providing incident light to the wound; and
receiving light reflected from the wound, the light reflected from the wound including information relating to the viability of tissue within the wound.

9. A treatment system comprising:
a dressing;
a light receptor; and
an excitation light source;
wherein, when the wound dressing is deployed on a wound:
the light source provides light that propagates through the wound; and
the light receptor for receiving the transmitted light, the transmitted light including information relating to the viability of tissue within the wound.

10. A wound vacuum comprising:
a sponge for application to a wound;
a flexible sheet for covering and sealing the wound;
a plurality of bladders, each of said bladders in fluid communication with a portion of the sponge, each of said bladders containing a debridement substance;
a negative pressure device that applies negative pressure via a tube to the sponge, said negative pressure device being adapted to apply negative pressure via suction through the tube in order form a seal between the flexible sheet and the wound;
a light source for propagating light through the wound; and
an optical receptor for receiving light exiting the wound, the light exiting the wound including information relating to the viability of tissue within the wound.

11. A method comprising:
deploying a wound dressing on a wound, the wound dressing comprising a light receptor;
propagating light through the wound; and
using the optical receptor to receive light exiting the wound, the light exiting the wound including information relating to the viability of tissue within the wound.

12. A method for treating a wound comprising:
applying a sponge to a wound;
sealing the wound with a flexible sheet;
reducing a gas pressure adjacent the wound;
propagating light through the wound; and
receiving the light exiting the wound, the light exiting the wound including information relating to the viability of tissue within the wound.

13. Apparatus for treating a wounded body portion, the apparatus comprising:
a dressing supporting a light source, the light source for propagating light into at least a portion of the wounded body portion; and
an acoustic receiver for receiving acoustic energy from the wounded body portion;
wherein the acoustic energy is generated by interaction of the light with the wounded body portion.

14. A method for treating a wounded body portion, the wounded portion including an exposed surface, the method comprising:
generating a negative pressure region contiguous with the exposed surface;
propagating light across the surface; and
receiving from the wounded portion acoustic energy formed by the interaction of the light with the body portion.

15. Apparatus for treating a wounded body portion, the apparatus comprising:
a dressing supporting a light source, the light source for propagating light into at least a portion of the wounded body portion;
an optical receiver for receiving light scattered by the wounded body portion;
and an analytical module in communication with the receiver, the analytical module configured to output a signal indicative of necrotic tissue.

16. A method for treating a wounded body portion, the wounded body portion including an exposed surface, the method comprising:
generating a negative pressure region contiguous with the exposed surface;
propagating light across the surface into the wounded body portion;
receiving light scattered by the wounded body portion; and
generating a signal having a magnitude that is dependent on an amount of necrotic tissue in the portion of the wound.

17. Apparatus for treating a wounded body portion, the apparatus comprising:
a dressing supporting a light source, the light source for propagating light into at least a portion of the wounded body portion; and
an optical receiver for receiving scattered light exiting a surface of the wounded body portion, the scattered light including a first wavelength and a second wavelength, there being a difference between the first wavelength and the second wavelength, the difference being indicative of the motion of a fluid relative to solid tissue in the wounded body portion.
and an analytical module in communication with the receiver, the analytical module configured to output a signal indicative of necrotic tissue in the wounded body portion.

18. A method for treating a wounded body portion, the wounded portion including an exposed surface, the method comprising:
generating a negative pressure region contiguous with the exposed surface;
propagating light across the surface;
receiving scattered light exiting the wounded body portion; and
identifying in the scattered light a difference between a first wavelength and a second wavelength, the first wavelength scattered by a solid tissue and a second wavelength scattered by a fluid in motion with respect to the solid tissue, the solid tissue and the fluid being present in the wounded body portion.
